(19) United States
(12) Patent Application Publication
(10) Pub. No.: US 2017/0215728 A1
(43) Pub. Date: Aug. 3, 2017

(54) APPLANATION TONOMETER

(71) Applicant: University of Washington, Seattle, WA (US)

(72) Inventors: Joanne Wen, Seattle, WA (US); Alex T. Mariakakis, Seattle, WA (US); Edward J. Wang, Seattle, WA (US); Neil E. Warren, Lynnwood, WA (US)

(21) Appl. No.: 15/422,226

(22) Filed: Feb. 1, 2017

Related U.S. Application Data
(60) Provisional application No. 62/289,755, filed on Feb. 1, 2016.
(60) Provisional application No. 62/375,779, filed on Aug. 16, 2016.

(51) Int. Cl. A61B 3/16; A61B 3/00; A61B 3/14

(52) U.S. Cl. CPC A61B 3/16; A61B 3/14; A61B 3/0025; A61B 3/0041

(57) ABSTRACT

Intraocular pressure measurement devices and techniques for use therewith are generally described. In some examples the pressure measurement device may include a sleeve comprising a lower end including a first opening and an upper end. In various examples, a fastener may be used to secure the upper end of the sleeve to a device comprising an image sensor. In some examples the intraocular pressure measurement devices may comprise an applanator having an optically clear body, a first end effective to protrude from the first opening of the sleeve, and a tip at the first end. In some examples, the applanator may be slidably retained within the sleeve such that when the sleeve is positioned vertically above an eye of a subject, the applanator may slide downward until the tip of the applanator protrudes from the first opening of the sleeve and rests upon a surface of the eye.

### CROSS-REFERENCE TO RELATED APPLICATIONS

This application claims the benefit of U.S. Provisional Application No. 62/289,755, filed Feb. 1, 2016 and U.S. Provisional Application No. 62/375,779, filed Aug. 16, 2016, the disclosures of which are incorporated herein by reference in their entirety.

### FIELD

This application relates to ophthalmology and, more specifically, to tonometry.

### BACKGROUND

Tonometry is a procedure for measuring intraocular pressure (IOP) inside the eye. Tonometry is often used to evaluate patients at risk for glaucoma. Tonometry flattens or applanates an area of the cornea of the subject. If a constant force is used to applanate the cornea, the intraocular pressure of the subject may be inferred from the size of the applanate region (e.g., the diameter or area of the applanate region). If a constant area is applanated, the intraocular pressure of the subject may be inferred from the amount of force used to applanate the constant area. Applanation tonometry is based on the Imbert-Fick law, which states that the pressure in a sphere filled with liquid and surrounded by an infinitely thin membrane is measured by the counter-pressure which just flattens the membrane.

### SUMMARY

In various examples, systems and methods are provided for applanation tonometry devices and techniques used to determine intraocular pressure of a subject's eye.

In accordance with some embodiments of the present invention, intraocular pressure measurement devices are generally described. In some examples, the intraocular pressure measurement devices may comprise a sleeve comprising a lower end and an upper end. In various examples, the lower end may comprise a first opening. In yet other examples, the intraocular pressure measurement devices may further comprise a fastener effective to secure the upper end of the sleeve to a device comprising an image sensor. In still other examples, the intraocular pressure measurement devices may further comprise an applanator having an optically clear body, a first end effective to protrude from the first opening of the sleeve, and a tip at the first end. In some examples, the applanator may be slidably retained within the sleeve such that when the sleeve is positioned vertically above an eye of a subject, the applanator may slide downward until the tip of the applanator protrudes from the first opening of the sleeve and rests upon a surface of the eye.

In accordance with some other embodiments of the present invention, methods of measuring intraocular pressure are generally described. In some examples, the methods of measuring intraocular pressure may comprise attaching a pressure measurement device to a mobile computing device comprising an image sensor. In various examples, the pressure measurement device may comprise a sleeve and an applanator slidably retained within the sleeve. In some examples, the sleeve may comprise a lower end and an upper end. In various further examples, the lower end may comprise a first opening and an interior of the sleeve may be aligned with a lens of the image sensor. In some further examples, the methods of measuring intraocular pressure may further comprise applying the applanator to an eye of a subject such that a flat tip of the applanator rests upon a surface of the eye to produce an applanated zone in the eye. In still further examples, the methods of measuring intraocular pressure may comprise capturing an image of the applanated zone through the applanator using the image sensor of the mobile computing device. In various other examples, the methods of measuring intraocular pressure may further comprise evaluating the image of the applanated zone using a processor of the mobile computing device to determine an intraocular pressure of the eye of the subject.

In accordance with some other embodiments of the present invention, other intraocular pressure measurement devices are generally described. For example, these intraocular pressure measurement devices may comprise a sleeve comprising a lower end and an upper end. In some examples, the lower end may comprise a first opening. In yet other examples, the intraocular pressure measurement devices may comprise a fastener effective to secure the upper end of the sleeve to a device comprising an image sensor. In still other examples, the intraocular pressure measurement devices may further comprise an applanator having an optically clear body, a first end effective to protrude from the first opening of the sleeve, and a tip at the first end. In some examples, the applanator may be slidably retained within the sleeve such that the first end of the applanator may be effective to slide out of the first opening to allow the tip to contact a surface of an eye of a subject. In various further examples, the intraocular pressure measurement devices may further include a light guide effective to direct light received from a light source toward the lower end of the sleeve.

Still other embodiments of the present invention will become readily apparent to those skilled in the art from the following detailed description, wherein are described embodiments by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF DRAWINGS

FIG. 1 depicts a side, cross-sectional view of an applanation tonometer device secured to a mobile computing device, in accordance with various embodiments of the present invention.

FIG. 2 depicts an isometric view of an applanation tonometer device secured to a mobile computing device case, in accordance with various embodiments of the present disclosure.

FIG. 3 depicts an example fastener for securing an applanation tonometer device to a mobile computing device, in accordance with some aspects of the present disclosure.

FIG. 4 illustrates a perspective view of an applanation tonometer device including a light path, in accordance with various aspects of the present disclosure.

FIG. 5 illustrates a perspective view of an applanation tonometer device including a light path and a fastener for securing the applanation tonometer device to an image sensor device and/or a light source, in accordance with various aspects of the present disclosure.

FIG. 6 depicts a computer implemented process for determining intraocular pressure of a subject's eye based upon an input image of an applanation zone on the subject's eye, in accordance with various aspects of the present disclosure.

FIG. 7 depicts an example of a computer vision technique for ellipse detection, in accordance with various aspects of the present disclosure.

FIG. 8 depicts an example process for measuring intraocular pressure using an applanation tonometer device, in accordance with various aspects of the present disclosure.

FIG. 9 depicts an example computing device effective to perform the various processing techniques described herein.

### DETAILED DESCRIPTION

In the following description, reference is made to the accompanying drawings that illustrate several embodiments of the present disclosure. It is to be understood that other embodiments may be utilized and system or process changes may be made without departing from the spirit and scope of the present disclosure. The following detailed description is not to be taken in a limiting sense, and the scope of the embodiments of the present invention is defined only by the claims of the issued patent. It is to be understood that drawings are not necessarily drawn to scale.

Various embodiments of the present disclosure provide improved systems and methods for portable applanation tonometry of an eye of a subject. These embodiments may provide a portable and inexpensive means of testing intraocular pressure in non-clinical environments, as well as overcoming various technical challenges presented through other means of estimation of applanation diameter (e.g., human estimation error). The computer vision techniques described herein, as well as the optically clear applanator of the applanation tonometer device allows for repeatable, accurate, and precise results.

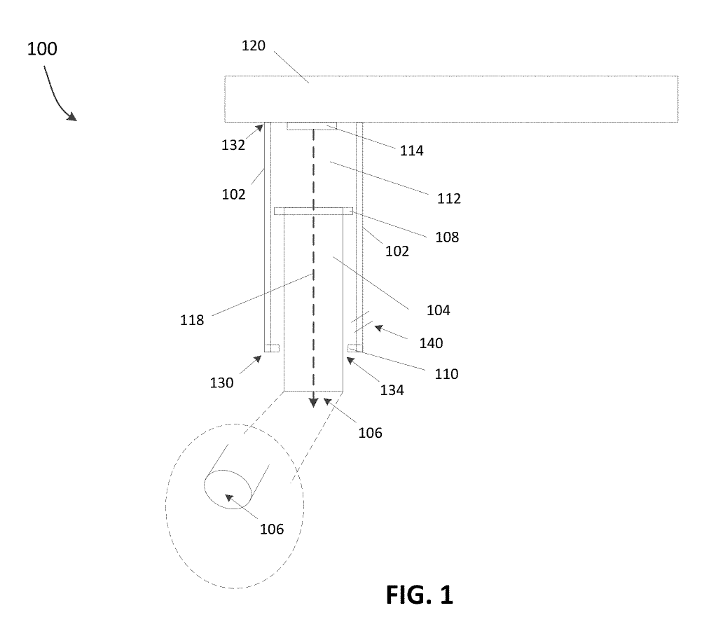

FIG. 1 depicts a side, cross-sectional view of an applanation tonometer device 100 secured to a mobile computing device 120, in accordance with various embodiments of the present invention. Applanation tonometer device 100 may include a sleeve 102. Sleeve 102 is depicted in cross section in FIG. 1. In some examples, sleeve 102 may partially or wholly comprise an opaque material effective to prevent various wavelengths of light from passing through surfaces of sleeve 102.

In some examples, sleeve 102 may be an elongate and cylindrical tube-like structure with a hollow interior 112. In various other examples, sleeve 102 may be non-elongate. In some examples, sleeve 102 may comprise a cubic or other parallelepiped-shaped structure. Additionally, various other shapes of sleeve 102, apart from those described above, may be used in accordance with the present disclosure.

Sleeve 102 may comprise a first end 130 and a second end 132. First end 130 of sleeve 102 may comprise an opening 134. Second end 132 of sleeve 102 may be effective to be optically aligned with a lens of image sensor 114. In some examples, an intervening structure or structures may be disposed between second end 132 and the image sensor 114. Second end 132 of sleeve 102 may be effective to be optically aligned with a lens of image sensor 114 along optical axis 118. In various examples, second end 132 of sleeve 102 may be covered by a transparent material and/or by a lens and/or light filter.

Image sensor 114 may be a camera image sensor such as a CCD (charge-coupled device) and/or a CMOS (complementary metal-oxide semiconductor) imager. In some examples, image sensor 114 may be integrated into a mobile computing device such as a smart phone, tablet computing device, lap-top computer, wearable computing device, and/or other mobile computing device. As is described in further detail below, image sensor 114 may be effective to capture image data representing objects positioned proximate to the first end 130 of sleeve 102. For example, image sensor 114 may be effective to capture image data representing an eye of a subject using applanation tonometer device 100. As described in further detail below, the image data representing the subject's eye may, in turn, be used by a computing device (e.g., mobile computing device 120) to determine the intraocular pressure of the subject's eye. As described herein, image data may refer to both single frames of image data as well as video data comprising multiple frames of image data. To detect image data, image sensor 114 may detect light reflected from objects outside of sleeve 102. The reflected light may travel along optical axis 118 through an applanator 104, through the hollow interior 112 of sleeve 102, to image sensor 114. Image sensor 114 may receive the reflected light and may convert the reflected light to image data.

In some examples, mobile computing device 120 and/or image sensor 114 may comprise a light source (not shown in FIG. 1) that may be positioned proximate to image sensor 114. In various examples, and as will be described in further detail below, sleeve 102 may comprise a separate channel for light emitted from such a light source. In various other examples, applanation tonometer device 100 may comprise a separate light guide positioned adjacent to sleeve 102 to guide and direct light emitted from a light source of mobile computing device 120. In some examples, an external light source that is not part of the mobile computing device or image sensor 114 may be used. In examples where applanation tonometer device 100 comprises a light guide that is separate from sleeve 102, an opaque material may be disposed between the light guide and the interior of sleeve 102 to prevent light passing through the light guide from entering sleeve 102. Light emitted from a light source of mobile computing device 120 may be directed through the light guide and/or through a separate channel of sleeve 102 to an area proximate to first end 130 of sleeve 102. In one illustrative example, light may be directed through a reflective passageway 140 formed adjacent to a wall or other exterior surface of sleeve 102. In another example, light may be directed through a fiber optic cable toward first end 130 of sleeve 102.

Applanator 104 may be sized and shaped so as to fit within the hollow interior 112 of sleeve 102. In some examples, applanator 104 may be sized and shaped such that, when sleeve 102 is oriented vertically (as shown in FIG. 1), applanator 104 may be pulled downward by gravity. The applanator 104 may be slidably retained within sleeve 102 such that at least a portion of applanator 104 may slide through opening 134 of first end 130 of sleeve 102 and may protrude from opening 134 of sleeve 102. In some examples, the applanator 104 may include a flange 108 to prevent the applanator 104 from completely falling out of the sleeve 102 when sleeve 102 is oriented vertically and/or in a downward direction. Similarly, first end 130 of sleeve 102 may include a flange 110 that may catch and/or abut flange 108 of applanator 104 to prevent applanator 104 from falling out of sleeve 102 through opening 134.

Applanator 104 may have a tip 106. The tip 106 may be a surface of applanator that is effective to protrude from applanator 104 when applanator 104 is actuated. In various examples, and as depicted within the dashed circle in FIG. 1, the tip 106 of applanator 104 may comprise a flat circular surface. In other examples, the tip 106 of applanator 104 may comprise a square, rectangular, elliptical, or other suitable shape for applanating a portion of a subject's cornea. As described above, in some examples, applanator 104 may be actuated by gravity by orienting the applanation tonometer device 100 vertically so that applanator 104 slides downward with at least a portion of the applanator 104 protruding from opening 134 of sleeve 102. In some other examples, applanator 104 may be actuated by a motor or other actuation mechanism of applanation tonometer device 100 to protrude from opening 134 with a specific force. In some examples, an active actuation mechanism (such as a motor) may be controlled via mobile computing device 120 and/or through an interface of the actuation mechanism itself (e.g., a mechanism such as a switch, toggle, button, touch sensor, etc. of the applanation tonometer device 100).

In some examples, tip 106 of applanator 104 may be optically clear and may be effective to cap the end of the applanator. In various further examples, tip 106 may be disposable and may be applied to applanator 104 before each use of applanation tonometer device 100 to reduce the risk of introducing contaminants to the surface of the subject's eye.

In examples where applanator 104 is gravity-actuated by orienting sleeve 102 vertically, applanator 104 may be of a known mass such that when sleeve 102 is vertically oriented the applanator 104 may be pulled downward by gravity through opening 134 with a constant, known force. Friction between portions of applanator 104 and the interior of walls of sleeve 102 may reduce the force exerted by applanator 104 accelerating downward due to gravity. Accordingly, in some examples, an accelerometer, gyroscope, and/or other position sensor of mobile computing device 120 may be used to determine when the applanation tonometer device 100 is vertically oriented. The mobile computing device 120 may provide an indication that sleeve 102 is vertically oriented in order to reduce the effects of friction between the walls of sleeve 102 and the applanator 104. For example, mobile computing device 120 may provide an audible and/or visual indication that sleeve 102 is vertically oriented.

Applanator 104 may be applied to the subject's cornea to flatten a portion of the cornea in order to determine the intraocular pressure of the subject's eye. For example, the applanation tonometer device 100 may be held over a supine subject such that the applanator 104 descends through sleeve 102 and at least partially protrudes from opening 134 of sleeve 102. In another example, the applanation tonometer device 100 may use an active actuation mechanism, such as a motor, to apply a known force to the subject's cornea with the tip 106 of applanator 104. In cases where an active actuation mechanism is used to apply a known force, the subject need not lay in a supine position.

The tip 106 of applanator 104 may contact the cornea of the subject's eye and may be sized and shaped so as to applanate a portion of the subject's cornea by applying a force to the cornea. The subject's intraocular pressure may be determined based on the size of the applanated area, when the cornea is applanated using a known force or forces. Applanator 104 may comprise a material that is at least partially optically clear so that light may pass through the applanator 104 and be detected by image sensor 114. Accordingly, image sensor 114 may capture an image of the subject's cornea (including any applanated portion thereof) along optical axis 118. Light may pass through the optically clear material of applanator 104 allowing images of the subject's eye to be captured through applanator 104. As will be described in further detail below, mobile computing device 120 may use the images to determine various characteristics regarding the applanated portion of the subject's cornea. For example, image processing software executed by mobile computing device 120 may be effective to determine a diameter of the applanated portion of the subject's cornea. Mobile computing device 120 may use the determined characteristic or characteristics of the applanated portion to determine an intraocular pressure of the subject.

In various examples, the applanator 104 may be a cylinder with a tip diameter of between 3 and 10 millimeters. Applanator 104 may comprise acrylic, glass, polycarbonate or some other optically clear material. The applanator 104 and/or sleeve 102 may have a length of between 40 and 70 millimeters. In some other examples, the applanator 104 and/or sleeve 102 may have a length between the upper and lower end of the applanator 104 and/or sleeve 102 of at least 60 millimeters. In some examples, the length of the applanator 104 may be chosen according to a focal length of the camera with which the applanation tonometer device 100 is to be used. In some examples, the applanator 104 may have a mass of between 3 and 8 grams. In various other examples, the applanator 104 may have a mass of between 3 and 15 grams. In various other examples, the applanator 104 may have a mass of 5.0 grams. In other examples, different masses may be used. The 5.0 gram mass may be advantageous because the conversion from applanation surface diameter to intraocular pressure has been clinically validated for human eyes for a mass of 5.0 grams. Conversion tables for larger masses are also available, however, studies have shown that increasing the weight of the tonometer may induce an increased pressure due to the displacement of aqueous humor during applanation.

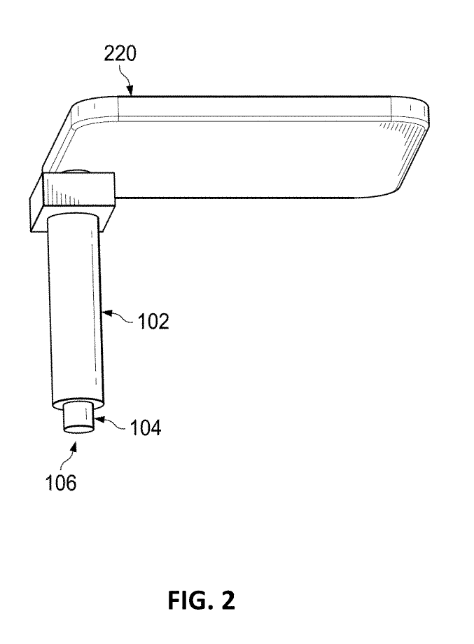

FIG. 2 depicts an isometric view of an applanation tonometer device secured to a mobile computing device case, in accordance with various embodiments of the present disclosure. In various examples, applanation tonometer device 100 may be integrated into a mobile computing device case 220 such that sleeve 102 is aligned with a lens of an image sensor device of a mobile computing device for which mobile computing device case 220 is designed. Accordingly, by inserting a compliant mobile computing device into mobile computing device case 220, the image sensor of the compliant mobile computing device may be optically aligned with the sleeve 102 and applanator 104 such that the image sensor may capture images through the applanator 104 of a subject's eye being applanated by applanator 104. Similarly, in examples where the applanation tonometer device includes an integrated light guide, the light source (e.g., flash) of the compliant mobile computing device may be aligned with the light guide of the applanation tonometer device by inserting the compliant mobile computing device into the mobile computing device case 220.

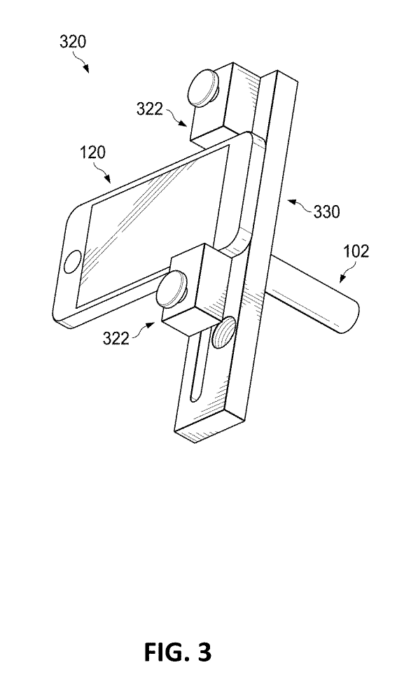

FIG. 3 depicts an example fastener 320 for securing an applanation tonometer device to a mobile computing device, in accordance with some aspects of the present disclosure. In various examples, fastener 320 may comprise a support structure 330 and one or more fastening mechanisms 322. Fastening mechanisms 322 may include clamps, elastic bands, grooves for holding the contours of a mobile computing device 120, a snap-in mold for mobile computing device 120 or another structure effective to secure mobile computing device 120 to sleeve 102 in such a way that images may be captured through sleeve 102 by an image sensor of mobile computing device 120. In various examples, support structure 330 may be formed in such a way as to not occlude an image sensor and/or light source of mobile computing device 120. For example, one or more openings may be formed in support structure 330 through which an image sensor of mobile computing device 120 may capture images and emit light (e.g. through a flash). Support structure 330 may be attached or otherwise coupled to sleeve 102 of the applanation tonometer device. In some other examples, a support structure 330 may not be used as a part of fastener 320 and sleeve 102 may directly contact mobile computing device 120 and/or a case of mobile computing device 120 while being secured in place by fastener 320.

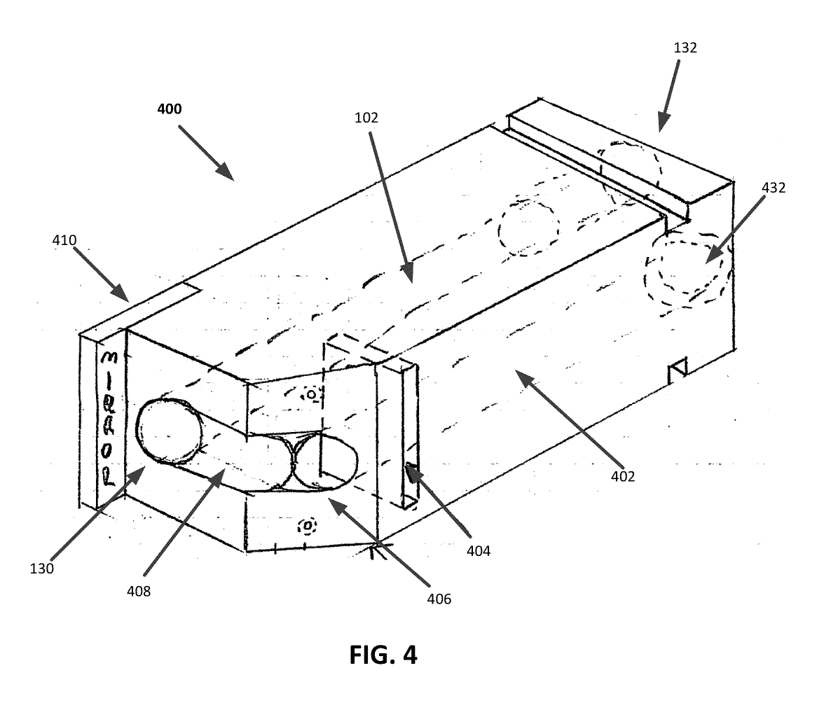

FIG. 4 illustrates a partially transparent, perspective view of an applanation tonometer device 400 including a light path, in accordance with various aspects of the present disclosure. Although, applanation tonometer device 400 is depicted with partially transparent structures in FIG. 4, the various surfaces of applanation tonometer device 400 need not be transparent, and are shown as such merely to illustrate various components of applanation tonometer device 400 which may otherwise be obstructed from view.

As depicted in FIG. 4, in some examples, the sleeve 102 and applanator 104 (not shown in FIG. 4) may be combined in applanation tonometer device 400 along with a light guide 402. The light guide 402 of structure 400 may be effective to direct light from a light source of a mobile computing device or other light source toward first end 130 of sleeve 102 so that an area proximate to the tip 106 of applanator 104 (depicted in FIG. 1) is illuminated via the light emitted from the light source.

In an example, light emitted from a light source of a mobile computing device may enter the light path of light guide 402 at opening 432. In some examples, opening 432 may be covered by a lens, light diffuser, collimator, or other optically transparent material. Light guide 402 may be a hollow tube or other-shaped pathway of applanation tonometer device 400. In some examples, the walls of light guide 402 may comprise a reflective material such that light is reflected internally within light guide 402 to facilitate light traveling from opening 432 to first end 130 of sleeve 102 and to reduce absorption of the light by the walls or other surfaces forming light guide 402. A transverse portion 406 of light guide 402 may include one or more mirrors to change the direction of light and/or direct the light toward first end 130 of sleeve 102. One or more mirrors in transverse portion 406 of light guide 402 may reflect light to portion 408 of light guide 402. A mirror 410 may be used to reflect and concentrate light toward first end 130 of sleeve 102. Accordingly, tip 106 of applanator 104 (depicted in FIG. 1) may be better illuminated by light from a light source of a mobile computing device. Illuminating the tip 106 (depicted in FIG. 1) of applanator 104 without introducing light from the external light source along the length of sleeve 102 may improve the quality of images of a subject's cornea captured through an optically clear applanator 104. Accordingly, opaque and/or reflective materials may be used between an interior of sleeve 102 and the interior of light guide 402 to prevent light from light guide 402 from passing through a wall of light guide 402 into the interior of sleeve 102.

In some examples, an optical filter 404 may be disposed along the light path of light guide 402 to filter light traveling through light guide 402. In some examples, a fluorescein dye may be placed on the eye of the subject to be tested. The fluorescein dye may fluoresce under blue light. Accordingly, optical filter 404 may comprise a material that selectively transmits blue light while absorbing other wavelengths of light. In some other examples other dyes may be used. Similarly, other optical filters may be used to selectively transmit other ranges of wavelengths of light depending on the wavelength(s) and/or color of light needed to cause the particular dye to fluoresce and/or needed to illuminate the eye of the subject.

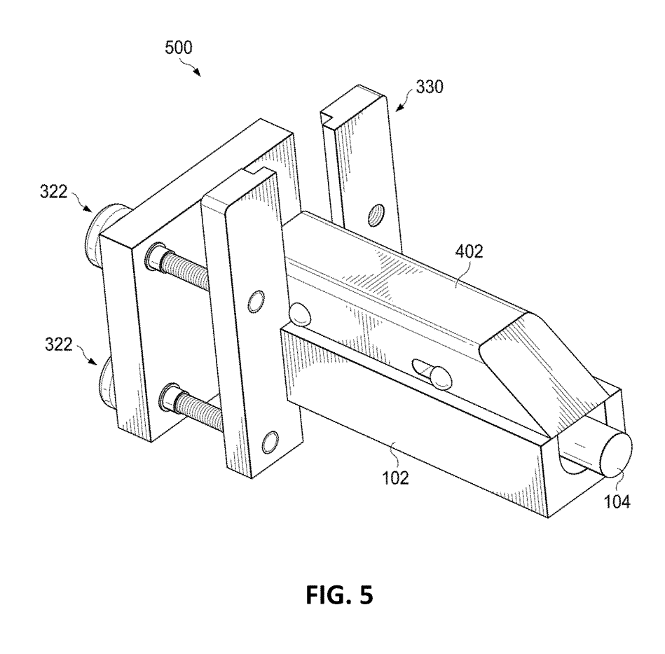

FIG. 5 illustrates a perspective view of an example applanation tonometer device 500 including a light path 402 and fasteners 322 for securing the applanation tonometer device 500 to an image sensor and/or a light source, in accordance with various aspects of the present disclosure.

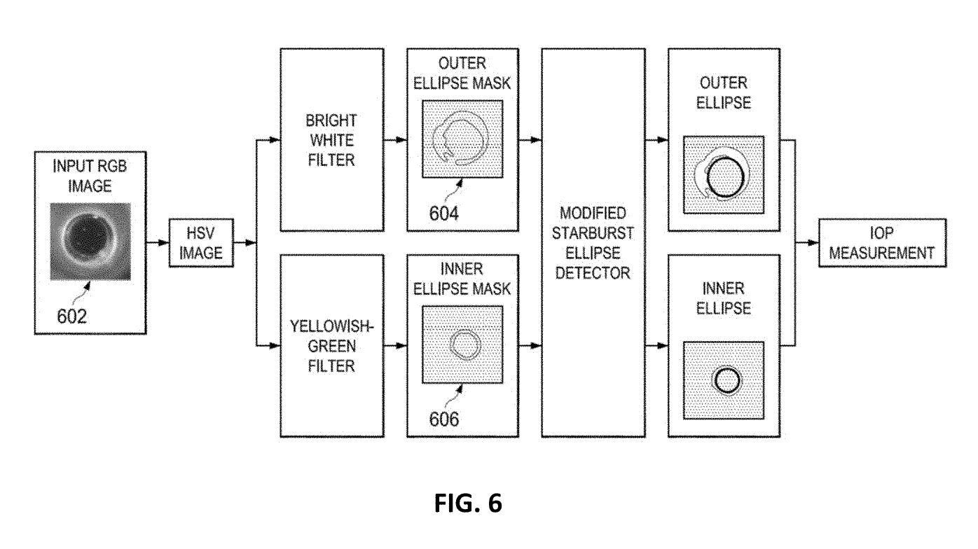

FIG. 6 depicts a computer implemented process for determining intraocular pressure of a subject's eye based upon an input image of an applanation zone on the subject's eye, in accordance with various aspects of the present disclosure. An example image processing technique for the various applanation tonometer devices of the present disclosure is described below with reference to FIG. 6.

To enhance the visibility of an acrylic or otherwise optically clear applanator for purposes of capturing images with an image sensor, the edge of the cylinder's bottom surface may be frosted. To emphasize the applanation surface for the image sensor, light may be directed toward the applanation surface using the various techniques described herein. Light may be filtered in order to produce a fluorescence of a dye applied to the subject's cornea (e.g., fluorescein dye).

Before receiving the assessment using the applanation tonometer device, the subject may assume a supine position if the applanation tonometer device uses gravity to produce the applanating force. Otherwise, if an active actuation mechanism is used to applanate the subject's cornea, the subject may assume any desired position. The user of the applanation tonometer device may administer a topical anesthetic with or without fluorescein dye (or another dye) to the subject's eye. The user may hold the applanation tonometer device over the subject's eye such that only the weight of the applanator is applied to the eye. This means that the applanation tonometer device should be as flat and/or as vertical as possible. As previously described, an accelerometer of a mobile computing device may be used as a kind of bubble-level to inform a reader when the applanation tonometer device is in the correct, vertically-oriented position.

The weight of a cylindrical applanator may create an elliptical applanation surface with a yellowish green outline (where fluorescein dye is used) when LED light or other light is shone on the subject's eye. The image sensor of the mobile computing device records the applanation of the subject's eye. The frames from the resulting video are processed using computer vision to give a real-time estimate of the subject's intraocular pressure, as described below.

A mobile computing device, such as mobile computing device 120 depicted in FIG. 1, may be effective to capture an image 602 of a subject's eye while the applanation tonometer device 100 is used to applanate a portion of the cornea. As described previously, in some examples, image 602 may represent a frame of a video captured by the image sensor. In some other examples, image 602 may represent a steady state produced by combining multiple image frames of a video captured by the image sensor, such as image sensor 114 described above with respect to FIG. 1.

In an example, the image 602 may be an RGB input image received from image sensor 114. The mobile computing device may execute a computer vision algorithm effective to detect two ellipses of input RGB image 602: 1) the outline of the applanator base in contact with the eye of the subject (i.e., the “outer ellipse”) and 2) the outline of the applanation surface (i.e., the “inner ellipse”). In some examples, the applanator 104 depicted in FIG. 1 may be cylindrical in shape. The diameter of the base of the optically clear cylinder (i.e., the base of the applanator) may be known, and so the applanation surface may be assigned an absolute measurement by using the cylinder diameter as a reference. Both the inner and outer ellipses will be relatively circular in examples where applanator 104 is cylindrical. However, the inner and outer ellipses may appear slightly elliptical if the hardware adapter is improperly mounted, and the applanation surface may appear elliptical if the subject has significant astigmatism and/or corneal surface irregularities.

The outer edge of input image 602, representing the edge of the outline of the applanator base may appear bright and white due to external illumination (e.g., from light received through light guide 402 depicted in FIG. 4 and/or from another light source). In some examples, the outer edge may appear bright and white due to light directed to the tip of the applanator by light guide 402 depicted in FIG. 4. As shown in FIG. 6, the outer ellipse mask 604 may be filtered using a bright white filter to emphasize the outer edge of input image 602. The inner edge representing the edge of the outline of the applanation surface may appear as a dimmer yellowish green where fluorescein dye is used. The input image 602 may be filtered according to intensity and color information to produce binary masks 604 and 606. The binary masks may represent the outlines of the inner and outer ellipses of input image 602. For example, an outer ellipse mask 604 may be produced representing the edge of the outline of the applanator base, and an inner ellipse mask 606 may be produced representing the edge of the outline of the applanation surface. The inner ellipse mask 606 and the outer ellipse mask 604 may be produced by, for example, converting image 602 into the hue-saturation-value color space (HSV). Inner ellipse mask 606 may bound the hue between 15-45%, with the saturation between 35-100%, and the value 15-100%. These thresholds encode the greenish-yellow fluorescence that appears due to the fluorescein dye applied to the subject's eye. The outer ellipse mask 604 may threshold the saturation between 0-20% and the value between 25-100%. These outer ellipse mask thresholds encode the light emitted from a light source of the mobile computing device (e.g., a flash of the mobile computing device) and detected around the circumference of the applanator when in contact with the subject's eye. Both inner ellipse mask 606 and outer ellipse mask 604 may be smoothed using morphological filtering operations to create contiguous contours.

Both inner ellipse mask 606 and outer ellipse mask 604 may have some non-uniform thickness due to the application of the dye and extraneous reflections within the applanator. The diameters of interest, for purposes of determining intraocular pressure, correspond to the innermost edges of the inner ellipse mask 606 and the outer ellipse mask 604. Through the use of standard circle/ellipse detection methods, many overlapping circles/ellipses or no circles/ellipses may be discovered in the outer ellipse mask 604 and inner ellipse mask 606, depending on the evenness of the masks. Additionally, only part of the applanation surface may be visible if the applanation surface overlaps with the sclera, which may make it more difficult to detect the fluorescein dye. Accordingly, a pupil contour detection algorithm based on the Starburst algorithm (D. Li, D. Winfield, and D. J. Parkhurst, “Starburst: A hybrid algorithm for video-based eye tracking combining feature-based and model-based approaches,” in 2005 IEEE Computer Society Conference on Computer Vision and Pattern Recognition (CVPR '05)—Workshops, 2005, vol. 3, pp. 79) may be employed.

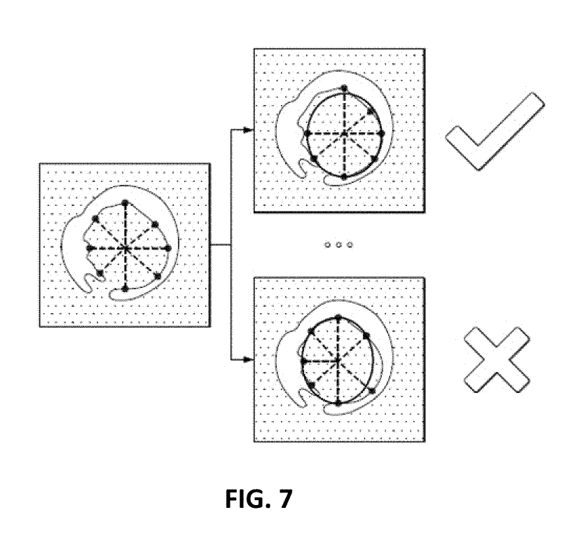

The ellipse detection method starts by assuming a reference point in the image. In some examples, the reference point may be chosen as the center of mass for the outer ellipse mask 604 given that the clear applanator should be centered within the image sensor's view. In various other examples, grid lines or other reference points with known dimensions may be used to select a reference point. As depicted in FIG. 7, the algorithm then steps radially at evenly spaced angles until an edge is reached in the outer ellipse mask 604. Alternatively, grid lines or other known reference points may be used instead of using the edge of the applanation surface as a reference. A number of pixels between the center reference point and the edge or other reference point of the outer ellipse mask 604 may be determined. Since the diameter of the applanation surface is known, a scaling ratio may be determined based on the number of pixels between the edge and the reference point and the known diameter. In other examples, where a reference point other than the edge of the applanation surface is used, grid lines may provide a known distance with which may be used to calculate the scaling ratio. Thereafter, the diameter of the inner-ellipse mask 606 (e.g., the applanation surface of the eye) may be determined based on the scaling ratio and the number of pixels between the reference point and the edge of the inner ellipse mask 606. This assumes that there are no contours that appear within the inner ellipse mask 606, which can happen for the applanation surface if the fluorescein pools in the subject's eye. Since accumulations of fluorescein may appear in the middle of the mask due to the distribution of the fluorescein, the radial steps start from a fixed distance just below the minimum expected radius to prevent the radial steps from stopping short.

Most of the detected edge points should belong to the desired ellipse, but some may still belong to artifacts along the edge of the contour. The original Starburst algorithm accounts for noisy ellipse points by fitting random subsets of points to ellipses and selecting the ellipse that minimizes the number of outliers. In the case of applanation, there is almost always a clean arc that appears in the image. Instead of randomized subsets of points, as used by Li et al., the proposed system fits contiguous subsets of points (three-quarters of the entire circumference) to ellipses. Although the base of the applanator cylinder and the applanation surface may appear elliptical, the ellipses should be relatively rounded. If the percent difference between an ellipses' major and minor axes is greater than 10%, it is automatically rejected by the algorithm. Amongst the rest of the ellipses produced by the different subsets of edge points, the ellipse that best fits the data according to Euclidean distance is selected. See, the example depicted in FIG. 7.

The ellipses recovered from the two masks (e.g., inner ellipse mask 606 and outer ellipse mask 604 depicted in FIG. 6) are then translated into circles with a radius equal to the average of the ellipses' axes. Given that the diameter of the applanator cylinder is known, the absolute measurement of the applanation surface can be recovered using the cylinder diameter as a reference. Every time an applanation is performed, a time series of diameter measurements is produced through image capture of the subject's eye with an image sensor of the mobile computing device. The measurements of interest can occur when the data is most stable, since that is when the weight of the applanator should be resting on the eye. Therefore, the system can combine, e.g., diameter measurements by taking a mean over the measurements within a desired standard deviation. For example, a standard deviation of 0.25 mm may be used over the course of 0.5 seconds. The final diameter measurement is mapped to an intraocular pressure value using a clinically validated lookup table, such as the one published by Adolph Posner (A. Posner, “Modified conversion tables for the Maklakov tonometer,” Eye. Ear. Nose Throat Mon., vol. 41, pp. 638-644, 1962.).

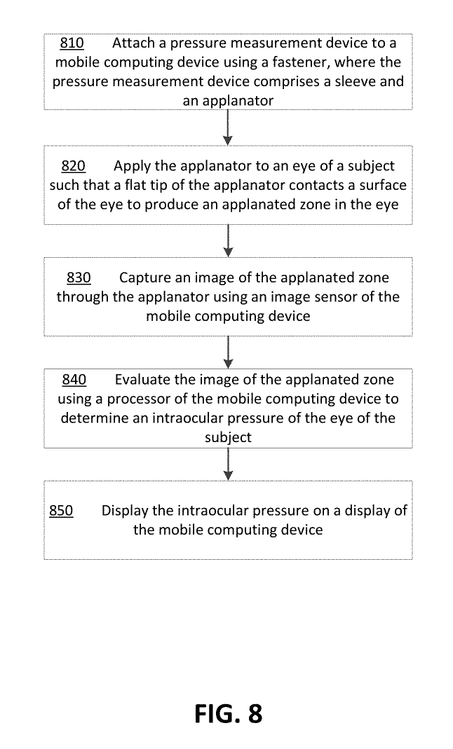

FIG. 8 depicts an example process for measuring intraocular pressure using an applanation tonometer device, in accordance with various aspects of the present disclosure. Those portions of FIG. 8 that have been described previously with respect to FIGS. 1-7 may not be described again for purposes of clarity and brevity.

The process in FIG. 8 may begin at action 810, “Attach a pressure measurement device to a mobile computing device using a fastener, where the pressure measurement device comprises a sleeve and an applanator”. At action 810 a pressure measurement device may be secured to a mobile computing device using a fastener. For example, fastener 320 depicted in FIG. 3 may be used to secure an applanation tonometer device 100 to a mobile computing device, such as mobile computing device 120 depicted in FIG. 1. In various examples, the fastener may include one or more clamps. In some other examples, the fastener may comprise a mobile computing device case. The pressure measurement device may comprise a sleeve, such as sleeve 102 from FIG. 1 and an applanator, such as applanator 104 described in FIG. 1. In examples where the fastener comprises a mobile computing device case, the sleeve may be molded to the mobile computing device case such that when the mobile computing device is installed in the mobile computing device case, a lens of the image sensor of the mobile computing device may be optically aligned with a longitudinal axis of the sleeve. In various examples, the applanator may be at least partially optically clear such that light may pass through the applanator. In some further examples, the pressure measurement device may be secured to the mobile computing device in such a way that an image sensor of the mobile computing device is optically aligned with a longitudinal axis of the sleeve so that the image sensor is able to capture images through the sleeve and through the at least partially optically clear applanator disposed within the sleeve.

The process in FIG. 8 may continue from action 810 to action 820, “Apply the applanator to an eye of a subject such that a flat tip of the applanator contacts a surface of the eye to produce an applanated zone in the eye.” At action 820, a portion of the applanator protruding from the sleeve may contact and applanate a portion of the subject's eye. In some examples, an active actuation mechanism may be used to produce a constant force with the applanator. In some other examples, gravity may be used to produce a constant force for an applanator with a known mass.

The process in FIG. 8 may continue from action 820 to action 830, “Capture an image of the applanated zone through the applanator using an image sensor of the mobile computing device, wherein the image is captured using light passing through the applanator.” At action 830, an image sensor of the mobile computing device may capture an image of the applanated zone through the applanator, which may be at least partially optically clear.

The process in FIG. 8 may continue from action 830 to action 840, “Evaluate the image of the applanated zone using a processor of the mobile computing device to determine an intraocular pressure of the eye of the subject.” At action 840 the image, which may include a single frame or an aggregated image comprising mean values of multiple image frames of a video, may be evaluated. For example, an input RGB image may be transformed into the HSV space and filtered to produce an outer ellipse mask (e.g., outer ellipse mask 604 from FIG. 6) and an inner ellipse mask (e.g., inner ellipse mask 606 from FIG. 6). The outer ellipse mask may represent the outline of the applanator contacting the surface of the subject's eye. The inner ellipse mask may represent the outline of the applanation zone. A reference point at the center of the outer ellipse mask may be used as a starting point to fit an ellipse to the inner edge of the circular image appearing in the outer ellipse mask. In some examples, a number of pixels between the center reference point and the inner edge of the circular image may be determined. Since the radius and diameter of the base of the applanator are known, the determined number of pixels may be converted into a distance. A second number of pixels between the center reference point and the inner edge of the inner ellipse mask, representing the applanation zone, may be determined. A diameter of the applanation zone may be determined by converting the second number of pixels into a distance (based on the per-pixel distance calculated from the known diameter of the applanator base). The diameter of the applanation zone may be converted into an intraocular pressure by using the diameter as the input to query a lookup table for a given applanation force. The lookup table may be stored in a memory of the mobile computing device or otherwise accessible by the mobile computing device.

The process in FIG. 8 may continue from action 840 to action 850, “Display the intraocular pressure on a display of the mobile computing device.” At action 850, after determining the intraocular pressure based on the diameter of the applanation zone, the intraocular pressure may be displayed on a display of the mobile computing device.

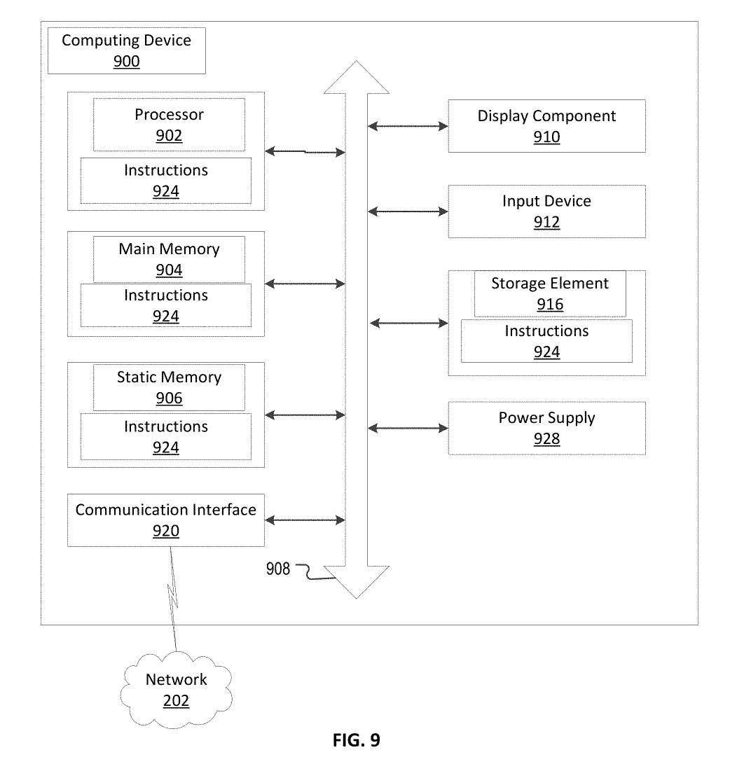

Referring to FIG. 9, the block diagram illustrates components of a computing device 900, according to some example embodiments, able to read instructions 924 from a non-transitory machine-readable storage medium (e.g., a hard drive storage system) and perform any one or more of the methodologies discussed herein, in whole or in part. Specifically, FIG. 9 shows the computing device 900 in the example form of a computer system within which the instructions 924 (e.g., software, a program, an application, an applet, an app, or other executable code) for causing the computing device 900 to perform any one or more of the methodologies discussed herein may be executed, in whole or in part. For example, the computing device 900 may be effective to execute the computer vision algorithms described above in reference to FIGS. 6-8.

In alternative embodiments, the computing device 900 operates as a standalone device or may be connected (e.g., networked) to other computing devices. In a networked deployment, the computing device 900 may operate in the capacity of a server computing device or a client computing device in a server-client network environment, or as a peer computing device in a distributed (e.g., peer-to-peer) network environment. The computing device 900 may include hardware, software, or combinations thereof, and may, as example, be a server computer, a client computer, a personal computer (PC), a tablet computer, a laptop computer, a netbook, a cellular telephone, a smartphone, a set-top box (STB), a personal digital assistant (PDA), a web appliance, a network router, a network switch, a network bridge, or any computing device capable of executing the instructions 924, sequentially or otherwise, that specify actions to be taken by that computing device. Further, while only a single computing device 900 is illustrated, the term “computing device” shall also be taken to include any collection of computing devices that individually or jointly execute the instructions 624 to perform all or part of any one or more of the methodologies discussed herein.

The computing device 900 includes a processor 902 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 904, and a static memory 906, which are configured to communicate with each other via a bus 908. The processor 902 may contain microcircuits that are configurable, temporarily or permanently, by some or all of the instructions 924 such that the processor 902 is configurable to perform any one or more of the methodologies described herein, in whole or in part. For example, a set of one or more microcircuits of the processor 902 may be configurable to execute one or more modules (e.g., software modules) described herein.

The computing device 900 may further include a display component 910. The display component 910 may comprise, for example, one or more devices such as cathode ray tubes (CRTs), liquid crystal display (LCD) screens, gas plasma-based flat panel displays, LCD projectors, or other types of display devices.

The computing device 900 may include one or more input devices 912 operable to receive inputs from a user. The input devices 912 can include, for example, a push button, touch pad, touch screen, wheel, joystick, keyboard, mouse, trackball, keypad, accelerometer, light gun, game controller, or any other such device or element whereby a user can provide inputs to the computing device 900. These input devices 912 may be physically incorporated into the computing device 900 or operably coupled to the computing device 900 via wired or wireless interface. For computing devices with touchscreen displays, the input devices 912 can include a touch sensor that operates in conjunction with the display component 910 to permit users to interact with the image displayed by the display component 906 using touch inputs (e.g., with a finger or stylus).

The computing device 911 may also include at least one communication interface 920, comprising one or more wireless components operable to communicate with one or more separate devices within a communication range of the particular wireless protocol. The wireless protocol can be any appropriate protocol used to enable devices to communicate wirelessly, such as Bluetooth, cellular, IEEE 802.11, or infrared communications protocols, such as an IrDA-compliant protocol. It should be understood that the communication interface 920 may also or alternatively comprise one or more wired communications interfaces for coupling and communicating with other devices.

The computing device 900 may also include a power supply 928, such as, for example, a rechargeable battery operable to be recharged through conventional plug-in approaches or through other approaches, such as capacitive charging. Alternatively, the power supply 928 may comprise a power supply unit which converts AC power from the power grid to regulated DC power for the internal components of the device 900.

The computing device 900 may also include a storage element 916. The storage element 916 includes the machine-readable medium on which are stored the instructions 924 embodying any one or more of the methodologies or functions described herein. The instructions 924 may also reside, completely or at least partially, within the main memory 904, within the processor 902 (e.g., within the processor's cache memory), or both, before or during execution thereof by the computing device 900. The instructions 924 may also reside in the static memory 906.

Accordingly, the main memory 904 and the processor 902 may also be considered machine-readable media (e.g., tangible and non-transitory machine-readable media). The instructions 924 may be transmitted or received over a network 202 via the communication interface 920. For example, the communication interface 920 may communicate the instructions 924 using any one or more transfer protocols (e.g., HTTP).

The computing device 900 may be implemented as any of a number of electronic devices, such as a tablet computing device, a smartphone, a media player, a portable gaming device, a portable digital assistant, a laptop computer, or a desktop computer. In some example embodiments, the computing device 900 may have one or more additional input components (e.g., sensors or gauges) (not shown). Examples of such input components include an image input component (e.g., one or more cameras), an audio input component (e.g., a microphone), a direction input component (e.g., a compass), a location input component (e.g., a GPS receiver), an orientation component (e.g., a gyroscope), a motion detection component (e.g., one or more accelerometers), an altitude detection component (e.g., an altimeter), and a gas detection component (e.g., a gas sensor). Inputs harvested by any one or more of these input components may be accessible and available for use by any of the modules described herein.

As used herein, the term “memory” refers to a non-transitory machine-readable medium capable of storing data temporarily or permanently and may be taken to include, but not be limited to, random-access memory (RAM), read-only memory (ROM), buffer memory, flash memory, and cache memory. The machine-readable medium is non-transitory in that it does not embody a propagating signal. While the machine-readable medium is described in example embodiments as a single medium, the term “machine-readable medium” should be taken to include a single medium or multiple media (e.g., a centralized or distributed database, or associated caches and servers) able to store instructions 924. The term “machine-readable medium” shall also be taken to include any medium, or combination of multiple media, that is capable of storing the instructions 924 for execution by the computing device 900, such that the instructions 924, when executed by one or more processors of the computing device 900 (e.g., processor 902), cause the computing device 900 to perform any one or more of the methodologies described herein, in whole or in part. Accordingly, a “machine-readable medium” refers to a single storage apparatus or device such as computing devices 110, 130, 140, or 150, as well as cloud-based storage systems or storage networks that include multiple storage apparatus or devices. The term “machine-readable medium” shall accordingly be taken to include, but not be limited to, one or more tangible (e.g., non-transitory) data repositories in the form of a solid-state memory, an optical medium, a magnetic medium, or any suitable combination thereof.

While the invention has been described in terms of particular embodiments and illustrative figures, those of ordinary skill in the art will recognize that the invention is not limited to the embodiments or figures described. For example, in various embodiments described above, a cylindrical, acrylic applanator is described. However, in other embodiments, the applanator may have another shape. Additionally, although in some examples described above a mobile computing device is described with an integrated image sensor and/or light source, in some examples, the light source and/or image sensor may be separate, stand-alone components.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one,” “at least one” or “one or more.” Unless otherwise required by context, singular terms used herein shall include pluralities and plural terms shall include the singular.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While specific embodiments and examples for the disclosure are described herein for illustrative purposes, various equivalent modifications are possible within the scope of the disclosure, as those skilled in the relevant art will recognize. Such modifications may include, but are not limited to, changes in the dimensions and/or the materials shown in the disclosed embodiments.

Specific elements of any embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Therefore, it should be understood that the invention can be practiced with modification and alteration within the spirit and scope of the appended claims. The description is not intended to be exhaustive or to limit the invention to the precise form disclosed. It should be understood that the invention can be practiced with modification and alteration and that the invention be limited only by the claims and the equivalents thereof.

What is claimed is:

1. An intraocular pressure measurement device, comprising:
a sleeve comprising a lower end and an upper end, wherein the lower end comprises a first opening;
a fastener effective to secure the upper end of the sleeve to a device comprising an image sensor; and
an applanator having an optically clear body, a first end effective to protrude from the first opening of the sleeve, and a tip at the first end, wherein the applanator is slidably retained within the sleeve such that when the sleeve is positioned vertically above an eye of a subject, the applanator slides downward until the tip of the applanator protrudes from the first opening of the sleeve and rests upon a surface of the eye.

2. The intraocular pressure measurement device of claim 1, wherein:
a second end of the applanator opposite the first end of the applanator comprises an applanator flange; and
the lower end of the sleeve comprises a sleeve flange configured to abut the applanator flange and retain the applanator within the sleeve.

3. The intraocular pressure measurement device of claim 1, wherein the sleeve comprises an opaque material.

4. The intraocular pressure measurement device of claim 1, wherein the upper end of the sleeve comprises a second opening and a transparent material covering the second opening.

5. The intraocular pressure measurement device of claim 1, wherein the device comprises a mobile computing device, wherein the mobile computing device comprises:
the image sensor;
a processor; and
a non-transitory computer-readable medium storing one or more instructions, which when executed by the processor are configured to cause the processor to perform a method comprising:
capturing an image of an eye of a subject;
determining a first number of pixels between a first reference point in the image and a second reference point in the image;
determining a scaling ratio based on a first distance between the first reference point and the second reference point and the first number of pixels;
determining a second number of pixels between the first reference point and a third reference point in the image, wherein the third reference point corresponds to an edge of an applanated portion of the eye of the subject;
determining a second distance between the first reference point and the third reference point based on the second number of pixels and the scaling ratio; and
determining a pressure of the eye of the subject based on the second distance.

6. The intraocular pressure measurement device of claim 5, wherein the second reference point represents a second edge of the tip of the applanator in the image.

7. The intraocular pressure measurement device of claim 1, further comprising a light guide positioned adjacent to the sleeve, the light guide effective to direct light from a light source to an area proximate to the first opening, wherein at least a portion of the light guide comprises an opaque material to at least partially prevent light traveling through the light guide from passing to an interior of the sleeve.

8. The intraocular pressure measurement device of claim 7, the light guide further comprising an optical filter effective to selectively transmit a range of wavelengths of light from the light source.

9. The intraocular pressure measurement device of claim 1, wherein the fastener comprises a mobile computing device case effective to hold a mobile computing device and align the upper end of the sleeve with a lens of the image sensor.

10. The intraocular pressure measurement device of claim 1, wherein:
the tip of the applanator comprises a flat circular surface having a diameter of between 3 and 10 mm.

11. The intraocular pressure measurement device of claim 1, wherein the applanator has a mass of between 3 and 15 grams.

12. The intraocular pressure measurement device of claim 1, wherein the tip of the applanator is optically clear and detachable from the applanator body.

13. A method of measuring intraocular pressure, the method comprising:
attaching a pressure measurement device to a mobile computing device comprising an image sensor, wherein the pressure measurement device comprises a sleeve and an applanator movably retained within the sleeve, the sleeve comprising a first end and a second end, wherein the first end comprises a first opening, and an interior of the sleeve is aligned with a lens of the image sensor;
applying the applanator to an eye of a subject such that a flat tip of the applanator contacts a surface of the eye to produce an applanated zone in the eye;
capturing an image of the applanated zone through the applanator using the image sensor of the mobile computing device; and
evaluating the image of the applanated zone using a processor of the mobile computing device to determine an intraocular pressure of the eye of the subject.

14. The method of claim 13, further comprising:
determining a first number of pixels between a first reference point in the image and a second reference point in the image;
determining a scaling ratio based on a first distance between the first reference point and the second reference point and the first number of pixels;
determining a second number of pixels between the first reference point and a third reference point in the image, wherein the third reference point corresponds to an edge of the applanated zone;
determining a second distance between the first reference point and the third reference point based on the second number of pixels and the scaling ratio; and
determining a pressure of the eye of the subject based on the second distance.

15. The method of claim 13, wherein the fastener comprises at least one clamp, and wherein securing the sleeve of the pressure measurement device to the mobile computing device further comprises clamping a support coupled to the sleeve to a mobile computing device using the clamp such that the image sensor is optically aligned with the first end and the second end of the sleeve.

16. The method of claim 13, wherein the fastener comprises a mobile computing device case effective to hold a mobile computing device, wherein the second end of the sleeve is molded to the mobile computing device case such that when the mobile computing device is installed in the mobile computing device case, the image sensor of the mobile computing device is optically aligned with the first end and the second end of the sleeve.

17. The method of claim 13, further comprising:
determining a diameter of the applanated zone using the image; and
querying a look-up table stored in a memory of the mobile computing device using the diameter to determine the intraocular pressure corresponding to the diameter, wherein the lookup table is associated with a mass of the applanator.

18. A intraocular pressure measurement device, comprising:
a sleeve comprising a lower end and an upper end, wherein the lower end comprises a first opening;
a fastener effective to secure the upper end of the sleeve to a device comprising an image sensor;
an applanator having an optically clear body, a first end effective to protrude from the first opening of the sleeve, and a tip at the first end, wherein the applanator is slidably retained within the sleeve such that the first end of the applanator is effective to slide out of the first opening to allow the tip to contact a surface of an eye of a subject; and
a light guide effective to direct light received from a light source toward the lower end of the sleeve.

19. The intraocular pressure measurement device of claim 18, wherein:
a second end of the applanator opposite the first end of the applanator comprises an applanator flange; and
the lower end of the sleeve comprises a sleeve flange configured to abut the applanator flange and retain the applanator within the sleeve.

20. The intraocular pressure measurement device of claim 18, wherein the sleeve comprises an opaque material.

21. The intraocular pressure measurement device of claim 18, further comprising a mobile computing device, wherein the mobile computing device further comprises:
the image sensor;
a processor; and
a non-transitory computer-readable medium storing one or more instructions, which when executed by the processor are configured to cause the processor to perform a method comprising:
capturing an image of an eye of a subject;
determining a first number of pixels between a first reference point in the image and a second reference point in the image;
determining a scaling ratio based on a first distance between the first reference point and the second reference point and the first number of pixels;
determining a second number of pixels between the first reference point and a third reference point in the image, wherein the third reference point corresponds to an edge of an applanated portion of the eye of the subject;
determining a second distance between the first reference point and the third reference point based on the second number of pixels and the scaling ratio; and
determining a pressure of the eye of the subject based on the second distance.
